(19) United States
(12) Patent Application Publication
(10) Pub. No.: US 2014/0074125 A1
(43) Pub. Date: Mar. 13, 2014

(54) Vertical Stitcher and Suturing Method

(71) Applicant: Hadi Piraka, Northville, MI (US)

(72) Inventor: Hadi Piraka, Northville, MI (US)

(21) Appl. No.: 13/610,804

(22) Filed: Sep. 11, 2012

(51) Int. Cl. A61B 17/062

(52) U.S. Cl. 606/147

(57) ABSTRACT

A surgical stitching device intended for suturing deep and difficult-to-reach wounds. It is a distinctive type of needle-holder designed to carry a straight needle alongside its main body. This instrument is distinguished from the currently available devices because it may not need laparoscopic visualization and sutures wounds from an inside out direction rather than from side-to-side.

### BACKGROUND

1. Field of the Invention

The present invention generally relates to surgical devices, and more particularly, to a new suturing instrument named “Vertical Stitcher” and a new surgical method called “Vertical Stitchery”. It is designed to close deep and difficult-to-reach surgical wounds

Its main applications are for closing deep tissue layers that are made through laparoscopy to prevent post-operative hernia formation. Laparoscopic procedures are widely used in surgery for diagnosing and treating medical conditions and diseases. Some patients may develop hernias at the specific sites of cuts in the abdomen which are made to perform laparoscopy.

This new invention could serve other purposes in which the vertically held needles are indicated herein and will be described later.

2 Related Technology

Currently, defects in the fascia, which are anatomically situated under the skin, are closed with stitches following completion of laparoscopy. Stitching is done either directly by exposing the fascia, or by using different devices that require direct laparoscopic visualization. Both approaches are rather difficult to perform, time consuming, and require quite an effort to achieve the desired result(s).

In overcoming the limitations and difficulties outlined above, the present invention provides for a surgical device that will make the deep and difficult-to-reach wounds significantly easier to close.

One of the main objectives related to this invention is closure of laparoscopic wounds. At the time of laparoscopy, small cuts are made in specific sites of the abdomen to facilitate insertion of a laparoscope and other instruments. These sites are known as “Laparoscopic Ports”. The incised skin is initially penetrated by a long rod with a pointed end, called “Trocar” and covered with a tubular cylinder named “Cannula”. As a consequence of these procedures, defects at the site of insertion of the trocar may develop in the fascia, which could result into future hernia formation.

There are potentially other indications for using the vertical stitcher such as: a) ligation (tying) of bleeding vessels at the port's sites which may happen as a result of blind insertion of the trocar- cannula; b) closure of the large abdominal incisions with a specific surgical technique and sutures called “Retention Sutures”, which is performed to prevent post-operative wound separation called “dehiscence”; and c) closure of the chest wall through approximation of the surgically separated ribs with the use of this new device, among other uses.

### SUMMARY

The present invention is based on a new stitching device which holds straight needles vertically or on an angle to its principle body structure, while conventional needle holders grasp curved needles horizontally.

Suturing of the deep tissue in tight surgical fields is achieved from inside to outside with this device, whereas conventional needle holders sutures the tissue from side-to-side.

Contrary to the conventional suturing technique, there is no need to visualize the deeply situated tissue with this new stitcher.

The design of the present invention and the method of suturing will simplify closure of deep, out-of-reach and difficult-to-visualize wounds, whereas regular needle holders could not serve the same purpose.

In one aspect, the present invention relates to a de novo surgical instrument and a different method of stitching. It is comprised of a modified regular needle holder or some other long clamps. Its jaws are partially rounded in a half circle or semi-circular fashion on one or both sides of the clamp to facilitate easy removal and insertion into deeply located wounds such as laparoscopic ports.

The jaws could have single action properties in which one of the two jaws open and closes, or double action, in which both jaws open and close simultaneously.

In another aspect of the present invention, two jaws are assembled—one on each side of the stitcher's distal end.

Surgical application of this device is carried on as the stitcher is loaded with a needle and thread (suture), it is pushed down directly through the laparoscopic port. As the depth of the needle's tip is felt to be under the fascia, based on the centimeter gauge at the shafts of the stitcher, it is pulled outward to allow penetration of this layer and to reach close to the skin. The skin now is retracted away with a small retractor and the needle is grasped with another clamp. The lock of the stitcher is now released as the needle is pulled out of the incision

This process is repeated on the other side of the incision and the suture ends on both sides of the fascia are tied under the skin. It is preferable to use a suture with double-armed needles for this purpose.

Alternatively, the needles may be passed through the entire abdominal wall including the skin (full thickness), and the sutures are then tied above the skin.

The stitcher as a whole, may be inserted through the laparoscopic cannula and suturing is accomplished similar to above, while the cannula is partially pulled out of the port.

The stitcher with double set jaws could be used to stitch both sides of the facial edges or full thickness of the abdomen as described above, but in one step.

The double set jaws, could also pass through a laparoscopic cannula and carry on suturing under direct laparoscopic visualization. It is preferable to be loaded with a double armed suture and to close the port wound in full thickness.

Both designs, single or double set jaws, require two or double armed straight needles with thread (suture)

Further objects, features, and advantages of this invention will become readily apparent to persons skilled in the art after a review of the following description, with references to the drawings and claims that are appended to and form a part of this specification.

### BRIEF DESCRIPTION OF THE DRAWINGS

FIG. 1 is a drawing of a single action jaw version of the stitcher

FIG. 1-1 is a segment of the jaw in FIG. 1 showing a long and slender slot designed for fixed housing position of the needle

FIG. 2 is a view of the closed jaws of the stitcher in FIG. 1.

FIG. 3 is a drawing of a stitcher with double-action jaws carrying a straight needle

FIG. 4 is a schematic drawing of the stitcher with twin sets of jaws

FIG. 5 is a cross section view of the tissue layers anatomically present in the abdominal wall

### DETAILED DESCRIPTION OF THE DRAWINGS

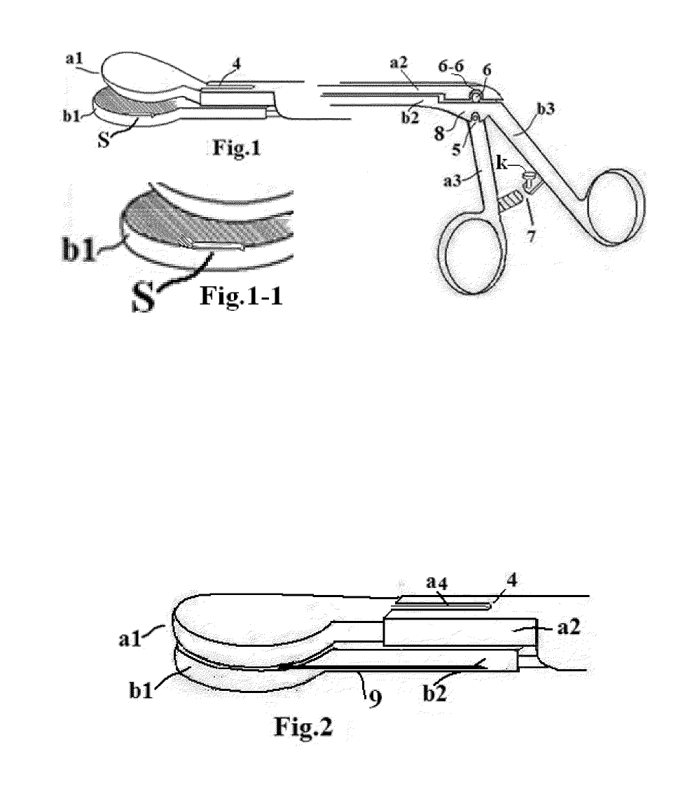

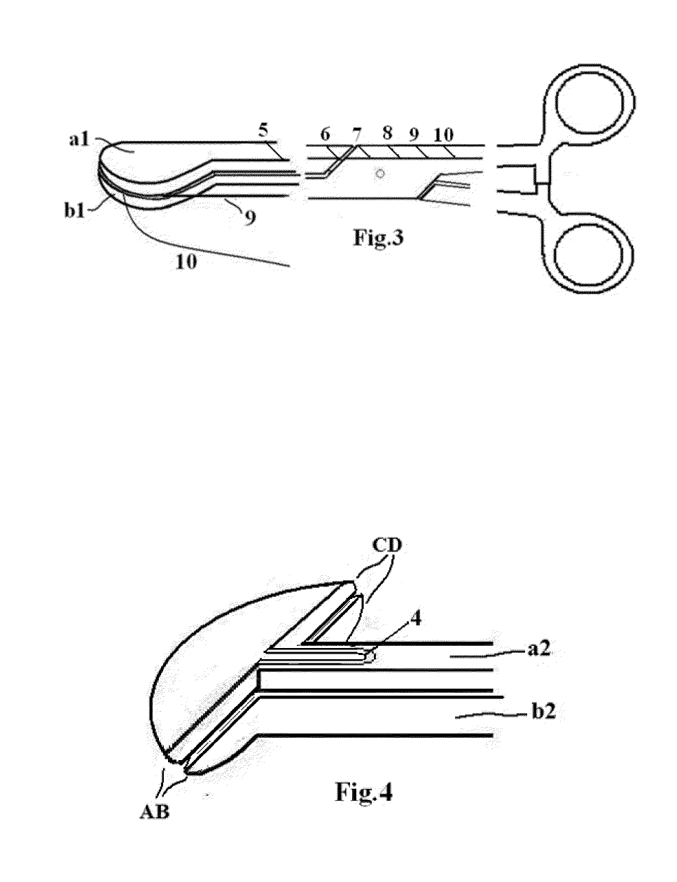

FIG. 1 is a view of the stitcher's main components consisting of a long clamp. This has fixed structure marked as b1, b2, and b3, and mobile assembly marked a1, a2, and a3. The jaws, al and b1, are configured in semi-circular formation on one side of the shaft for easy insertion and removal inside of deep wounds. It is modified in order to hold a straight needle. (As marked “9” in FIGS. 2 and 3)

The Jaw, a1, has the mobility to open and close, i.e. to hold or release the needle. This is accomplished by the swaying movement of the mobile segment of the shaft, a2, the hinge, 4, and the mobile handle, a3. This handle is connected to the shaft, a2, by a round, disc-like projection, 6, situated through a socket, 6-6 inside the shaft, b2. It is supported by a small pin, 5, and fixed into the shaft b2 while covered by an arc 8, in the proximity of the handle, b3.

A manual two-piece lock, 7, is secured at the distal end of the handles, which is released by pulling off the small knob, k, situated in one of its two plates. This piece is secured with a small spring at the base of its attachment to the handle, b3.

The inner surfaces of both jaws are grooved to create rough surfaces for strong holding of the needle, which could be loaded at variable angles.

FIG. 1-1 is a long slot, S, is secured near the outer edge of one of the two jaws for fixed positioning of the needle, if desired.

FIG. 2 views the closed jaws of the device illustrated in FIG. 1, to show the relationship between the fixed jaw, b1, and mobile jaw, a1. The stem, a4, of the mobile jaw, a1, is lodged in the hinge housing, 4. A needle, 9, is wedged between the jaws.

FIG. 3 is a drawing of the second version of the vertical needle holder comprises of double-action jaws. The semi-circular configuration of the jaws are included in this design as in FIG. 1 A straight needle, 9, is inserted between the jaws.

A gauge marked from five to ten centimeters is seen on one side of the device's shafts in FIG. 3, while the opposite side is not shown. This graduation is intended to specify the depth of the clamp in relation to the depth of the fascia

FIG. 4: Shows the third version of the stitcher which consists of a double set of jaws, AB and CD. This is designed to simultaneously suture the wound's edges in one step, rather than in two.

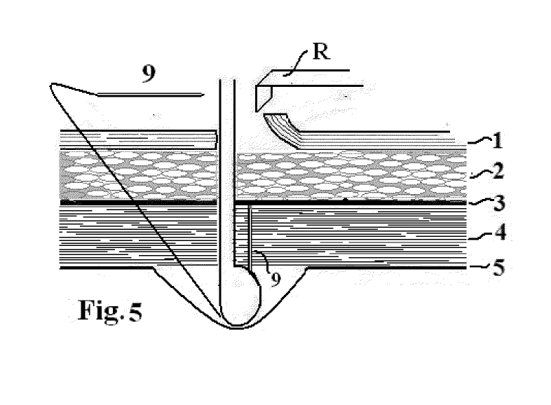

FIG. 5: Cross-section embodiment of various layers of the abdominal wall's tissues. These are: skin, marked as, 1, fatty tissue under the skin, 2, fascia, 3, muscles, 4, and the peritoneum, 5. A vertical stitcher with needle is passed through the track of a trocar and cannula

As a person skilled in the art will readily appreciate, the above description is meant as an illustration of implementation of the principles of this invention. This description is not intended to limit the scope or application of this invention in that the invention is susceptible to modification, variation and change, without departing from the spirit of this invention, as defined in the following claims.

What is claimed:

1. A surgical suturing device, the devise comprising:
a needle-holding clamp, two handles, a shaft, a lock, and two needle-grasping units (jaws);
a shaft having two components, mobile and fixed which in turn terminate into mobile and fixed jaws. The mobile part slides over the fixed components in order to open and close its corresponding hinged jaw for holding and/or releasing needles. The jaw's tips are rounded in a semi-circular fashion and protrude on a right angle to the shaft in order to hold straight needles. The needles are held parallel to or at an angle to the clamp's main body. The handles consist of fixed and mobile units too and are bent on an angle to provide better grips for the fingers;
a lock is installed between the handles and is released by pulling off a small pin attached at the edge of one of its two straps. The releasing component is secured with a spring at the base of its attachment to the clamp's handle;
the shaft may be marked and numerated on its both sides (not shown) for gauging the depth of the wound and position of the needle's tip in relation to the wound;
a long slot is secured near the outer edge of one of the two jaws for fixed positioning of the needle, if desired.

2. A surgical suturing device, the device comprising:
modified regular straight clamps or forceps with straight handles;
the jaws at the tip of the clamp are rounded in a semi-circular fashion to protrude on one side of the shaft in order to hold vertical needles parallel to the shaft and have double-action properties. They open and close simultaneously as they grasp or release the needle;
its shaft may be marked and numerated on both sides for gauging the depth of the wound and position of the needle's tip in relation to the intended wound;
a lock is installed in between the handles and is released by pulling off a small pin attached near the edge of one of its two straps. The releasing component of the lock is secured with a small spring at the base of its attachment to the clamp's handle;
a long slot is secured near the outer edge of one of the two jaws for fixed positioning of the needle, if desired.

3. A surgical suturing device, the devise comprising:
a stitcher consisting of double-headed jaws;
the jaws at the tip of the clamp are rounded in a semi-circular manner to protrude on both sides of the shaft;
its shaft may be marked and numerated on its both sides for gauging the depth of the wound and position of the needle's tip in relation to the intended wound;
a lock is installed in between the handles and is released by pulling off a small pin attached near the edge of one of its two straps. The releasing component of the lock is secured with a small spring at the base of its attachment to the clamp's handle;
a long slot is secured near the outer edge of one of the two jaws for fixed positioning of the needle, if desired.
